(19) United States
(12) Patent Application Publication
(10) Pub. No.: US 2024/0112819 A1
(43) Pub. Date: Apr. 4, 2024

(54) METHOD FOR ANALYZING POTENTIAL ABLATION THERAPIES

(71) Applicant: CathVision ApS, Kopenhangen (DK)

(72) Inventors: Rune Tore PAAMAND, Humlebaek (DK); Mads Emil MATTHIESEN, Genarp (SE)

(21) Appl. No.: 18/276,193

(22) PCT Filed: Dec. 3, 2021
(86) PCT No.: PCT/EP2021/084257

§ 371 Date: Aug. 7, 2023

(30) Foreign Application Priority Data
Feb. 11, 2021 (EP) 21156619.5

(51) Int. Cl. G16H 50/70; A61B 5/00; A61B 5/346; A61B 34/10; G16H 10/60

(52) U.S. Cl. CPC G16H 50/70; A61B 5/346; A61B 5/7267; A61B 34/10; G16H 10/60

(57) ABSTRACT

The invention relates to a method for analyzing potential ablation therapies (1), in particular for patients (P) with atrial fibrillation, via a control system (2), wherein the method comprises an analysis step (3) in which the control system (2) applies a trained machine learning model (4) to input data (5) thereby generating output data (6), wherein the input data (5) comprise electrical biomarkers (7) derived from ECG data (8) from a patient (P) of at least one potential ablation therapy (1), the potential ablation therapy (1) including at least one potential ablation event (9) with a potential ablation location (10), wherein the output data (6) comprise a predicted change of the electrical biomarkers (7) after applying the potential ablation therapy (1) to the patient (P), wherein the predicted change is derived from the input data (5) via the trained machine learning model (4).

### CROSS-REFERENCE TO RELATED APPLICATIONS

This application is a U.S. national stage of International Application Number PCT/EP2021/084257, filed Dec. 3, 2021, and claims priority to EP 21156619.5, filed Feb. 11, 2021.

### BACKGROUND OF INVENTION

The invention relates to a method for analyzing potential ablation therapies, to a computer readable medium with a trained machine learning model stored on it, to a control system configured to perform said method and to a surgery system.

The present method is particularly concerned with ablation therapy for atrial fibrillation. Electrically, atrial fibrillation is the chaotic activation of muscle cells of the atria. During atrial fibrillation, the atria only minimally contribute to the function of the heart. Atrial fibrillation, therefore, reduces the output of the heart but is not imminently dangerous. However, when becoming chronical, atrial fibrillation is correlated with increased morbidity and mortality. One treatment option for atrial fibrillation is ablation therapy. Ablation therapy is the destruction of the cells that allow electrical wave re-entry to reduce chaotic activation of the atrial muscle cells.

The success rate of ablation therapy depends on the location of the ablation. In many cases of atrial fibrillation, atria muscle cells around the pulmonary veins entry are ablated. While this standard therapy has shown good results for many patients, it is not always successful. For patients with complex atrial fibrillation or recurrent atrial fibrillation in particular, atrial mapping of the electrical wave fronts and re-entry points is available. However, such mapping is complex and hard to interpret. Further methods to identify and evaluate potential targets for ablation are therefore needed.

Known methods like EP 3 744 282 A2 use machine learning algorithms to generate a cardiac ablation treatment plan. However, applying machine learning to a great number of input variables requires a large training data set. It is further difficult to design a machine learning architecture that is able to generate a generic treatment plan from rather inhomogeneous input data. While considering a large number of input variables can in theory increase the precision of a machine learning algorithm, in practice, gathering the training data set from scratch by taking a large number of ablation therapies into account is unfeasible.

Other known methods (WO 2019/217430 A1) are concerned with learning activation pathways and treatment locations based on a measurement and functional models of the heart. A functional model is a good way to reduce the complexity of a machine learning algorithm and the required training data set. However, the functional understanding of atrial fibrillation is still progressing and combining functional models with machine learning algorithms is a complex task by itself.

### BRIEF SUMMARY OF THE INVENTION

It is therefore an object of the present invention to provide a method for analyzing potential ablation therapies with a manageable complexity that is realistically trainable and usable.

The above-noted problem is solved by the methods disclosed herein.

The main realization of the present invention is that pathologies of the heart that are treated with ablation are generally related to electrical misfunctions. As such, they are well described by the results of electrical measurements. It is therefore possible to develop a machine learning algorithm based on electrical data, namely ECG data. By deriving electrical biomarkers from ECG data and using a trained machine learning model to predict a change of the electrical biomarkers after applying a potential ablation therapy, the complexity of the machine learning algorithm is significantly reduced. In particular, by shifting the focus away from trying to learn a broadly defined overall success of a treatment using a multitude of data, the definition of a cost function for the training of the machine learning model becomes more easily manageable in the present invention. Being able to choose a good cost function is a key to successfully applying a machine learning algorithm.

A further advantage is that electrical measurements prior and posterior to ablation therapies are already available for a considerable number of patients. Deriving a predicted change of the electrical biomarkers also allows presenting the predicted change to a physician. This allows the physician to design an appropriate overall treatment for the patient based on the predicted change, paired with the physician's knowledge and experience. This can further enhance the quality of the overall treatment and the acceptance of the proposed method by physicians and patients.

In detail, a method for analyzing potential ablation therapies is proposed, in particular for patients with atrial fibrillation, via a control system, wherein the method comprises an analysis step in which the control system applies a trained machine learning model to input data thereby generating output data, wherein the input data comprise electrical biomarkers derived from ECG data from a patient of at least one potential ablation therapy, the potential ablation therapy including at least one potential ablation event with a potential ablation location, wherein the output data comprise a predicted change of the electrical biomarkers after applying the potential ablation therapy to the patient, wherein the predicted change is derived from the input data via the trained machine learning model.

The input data may be mainly constituted by ECG-derived data or ECG-derived data and ECG-data, further enhancing the above-mentioned advantages. The possibility of analyzing the output data to identify potential ablation targets for the patient is also disclosed. A predicted change in electrical biomarkers can be a good indicator for identifying potential ablation targets and planning an ablation therapy.

A training step for the machine learning model is proposed herein. The training data set may be derived from a training database which can be retrospectively labelled. This database may be based on an open or commercial database thereby greatly increasing the usability of the proposed method.

In an embodiment, the electrical biomarkers may be derived by the control system from the ECG data of the training database.

Presently, the biomarker response to single ablation events and the whole ablation therapy is of particular interest. In another embodiment, a first model for deriving a change of the electrical biomarkers after applying one of the potential ablation events and/or a second model to derive a change of the electrical biomarkers may be trained in the training step. In some cases, single ablation events may have a well-defined biomarker response while in other cases the biomarker response of the ablation therapy is composed by more than one ablation event. For the latter, the single ablation events may have a synergetic effect which can be seen by considering the whole ablation therapy.

The possibility that the first and the second model may be applied separately or jointly and in particular may be applied to different electrical biomarkers is disclosed herein.

In another embodiment, the training data set may comprise focal source ablation events. Focal source ablations as a result of mapping are more specific, provide a wider range of ablation locations and enable identifying potential focal sources by the proposed method.

A deriving step for deriving electrical biomarkers from the ECG data automatically is disclosed. The electrical biomarkers may be derived from the ECG data by non-machine learning algorithms . Here, known algorithms may be used to derive a number of electrical biomarkers from ECG data. In particular, those named herein are applied in the proposed method. However, it is equally preferred to use a machine learning model to derive one or more of the electrical biomarkers. This machine learning model could be part of the machine learning model for deriving the predicted change of the electrical biomarkers, thereby enabling the predictions based on ECG data directly.

In another embodiment, in particular depending on the available training data set, the possibilities of using mainly the last ablation event of the ablation therapies of the training database or using ablation events throughout the ablation therapies is considered.

Another embodiment relates to determining a classification of the potential ablation therapy by using the output data, which can also be based on a machine learning algorithm. Further, the analysis of electrical biomarkers based on non-machine learning algorithms can also be utilized in the classification.

The method may provide the output of a classification of potential ablation therapies, in particular from online ECG data measurements. The output can be used to identify or verify potential ablation locations even during surgery.

Another teaching disclosed herein, which is of equal importance, is directed to a computer readable medium with a trained machine learning model stored on it. All explanations given with respect to the proposed method and in particular with respect to the training step are fully applicable.

Another teaching, which is also of equal importance, is directed to a control system configured to perform the proposed method. All explanations given with respect to the proposed method are fully applicable.

Another teaching, which is also of equal importance, is directed to a surgery system connected to or forming part of the proposed control system. All explanations given with respect to the proposed method are fully applicable.

### BRIEF DESCRIPTION OF THE DRAWINGS

In the following, an embodiment of the invention is explained with respect to the drawings. The drawings show:

FIG. 1 the proposed method schematically;

FIG. 2 the training of the machine learning model; and

FIG. 3 the application of the trained machine learning model.

### DETAILED DESCRIPTION OF THE INVENTION

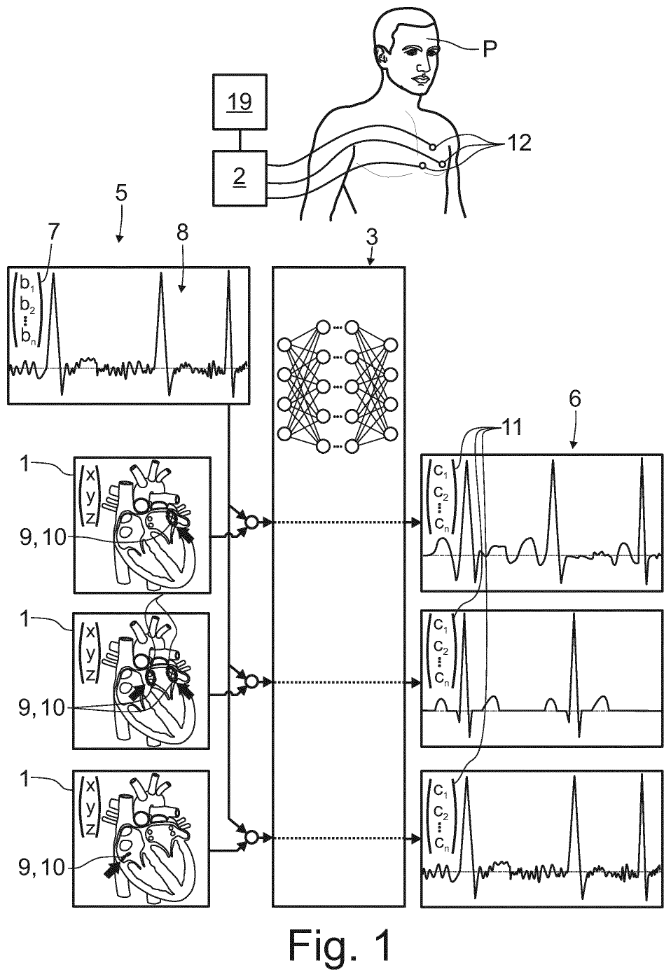

The proposed method is used for analyzing potential ablation therapies 1. In the preferred case shown in the Figures, the proposed method is used for patients P with atrial fibrillation. The potential ablation therapies 1 are analyzed via a control system 2. In FIG. 1, the control system 2 is shown schematically as a dedicated control system 2 comprising hardware connected to the patient P. The control system 2 may also be a general-purpose computer, a cloud-based computer system or the like and configured to perform the proposed method.

The method comprises an analysis step 3 in which the control system 2 applies a trained machine learning model 4 to input data 5 thereby generating output data 6.

Presently the term “trained machine learning model” describes at least the minimum amount of data necessary to apply the result of training of a machine learning algorithm. The trained machine learning model 4 may be present in a compressed representation and does not have to be usable by any general-purpose computer. The trained machine learning model 4 may, for example, comprise weights of an otherwise predefined artificial neural network. The necessary operations to apply the trained machine learning model 4 may be stored in the control system 2 and/or be part of the trained machine learning model 4. In a preferred embodiment, however, the trained machine learning model 4 comprises program code and is applicable to the input data 5 by a general-purpose operating system.

The input data 5 comprise electrical biomarkers 7 derived from ECG data 8 from a patient P of at least one potential ablation therapy 1. The potential ablation therapy 1 includes at least one potential ablation event 9 with a potential ablation location 10. The input data 5 are schematically shown on the left side in FIG. 1. The electrical biomarkers 7 will be explained further below in more detail. The potential ablation therapy 1 may comprise only one potential ablation event 9 or more than one potential ablation event 9. The potential ablation therapy 1, the potential ablation event 9 and the potential ablation location 10 may be stored in any suitable data structure. This data structure is preferably standardized. In FIG. 1, the values x, y, z represent the respective potential ablation therapy 1, e.g. the coordinates of a potential ablation location 10 in the potential ablation therapy 1, and are shown as an example for illustrative purposes. FIG. 1 depicts three potential ablation therapies 1, one of them comprising two potential ablation events 9. The term “potential” means that this ablation therapy 1 does not necessarily have to be applied to a patient P. Presently, the proposed method rather supports making a prediction if applying this potential ablation therapy 1 to a patient P is promising.

An ablation event 9 is formed by one or more regions in an area targeting a common focal source or by one or more regions that otherwise share a common area, for example, the entrance of a pulmonary vein. An ablation event may for example be one application of a cryocatheter.

The output data 6 comprise a predicted change of the electrical biomarkers 7 after applying the potential ablation therapy 1 to the patient P. The predicted change is derived from the input data 5 via the trained machine learning model 4. In FIG. 1, the change is shown in a set of changed electrical biomarkers 11. Further, a change in the ECG data 8 is illustrated for explanatory purposes.

The ECG data 8 are preferably surface ECG data or intracardiac ECG data and may be measured by electrodes 12 connected to the control system 2.

The electrical biomarkers 7, or some of the electrical biomarkers 7, may be time dependent and/or time independent. By choosing electrical biomarkers 7 as the focus of the machine learning mechanism and looking at the predicted change of the electrical biomarkers 7, a number of machine learning architectures become particularly suitable for the proposed method. The following list of preferred architectures is to be understood as not being comprehensive.

The trained machine learning model 4 is derived by training in a generally known manner. The trained machine learning model 4 is part of a machine learning mechanism comprising training and application of the trained machine learning model 4.

The underlying architecture may be a fixed-input architecture. Preferably the fixed-input architecture is a convolutional neural network, a feed forward network, a deep residual network or a classification architecture such as a support vector machine or a Bayes classifier. The fixed input architecture may be coupled to an autoencoder.

To generate the fixed input, the ECG data 8 may be split into time windows of a fixed length comprising one or more QRS-complexes. Additionally or alternatively, the electrical biomarker 7 may be time independent. Convolutional networks may be used for 2D input data 5. However, 1D implementations may also be applied, being well-suited for time series. Depending on the input data 5, a feed forward network is preferable, which may form a particularly simple approach. In particular, if the input data 5 comprise long time series, solutions for the vanishing gradient problem like deep residual networks can be used. In general, the ECG data 8 prior to an ablation therapy 1 may have a time dependence with early and late events being equally important. The named classification architectures may in particular be advantageous for a larger number of correlated electrical biomarkers 7.

Also preferred are recurrent architectures, in particular recurrent neural networks, more preferably, architectures based on long short-term memory or gated recurrent units, which can for example be used depending on the length of the ECG data 8 and/or the electrical biomarkers 7. Another preferred architecture is an attention architecture, in particular a transformer architecture and/or a non-recurrent architecture with an attention mechanism. In case it is not known in which part of ECG data 8 the relevant information for predicting the success of a potential ablation therapy 1 may be located, it can be advantageous to use low frequencies of ECG data 8 and derive long sequences of electrical biomarkers 7. In particular, if relevant information is sparsely distributed, attention architectures are well-suited.

The cost function for training may be based on the known response to the ablation therapy 1, as will be further described. The response can be compensated by time distance to the ablation therapy 1 and/or ablation event 9. It is preferred to predict a long time change of the electrical biomarkers 7 and/or a direct change of the electrical biomarkers 7 shortly after the ablation therapy 1.

Here and preferably, the input data 5 are mainly constituted by ECG-derived data, in particular by the electrical biomarkers 7. Alternatively, the input data 5 may be mainly constituted by ECG-derived data and ECG data 8. The term “mainly” here and in the following means that the named data form the core of the respective data or the algorithm using the respective data but other data may also be present. This takes into account that it is possible to include additional data into a machine learning algorithm without greatly influencing the result. The definition of the term “mainly” is therefore to be understood as a functional definition.

The targeted use of non-electrical data 8 may be advantageous for the proposed method. The proposed method may comprise analyzing output data 6 to identify potential ablation targets for the patient P.

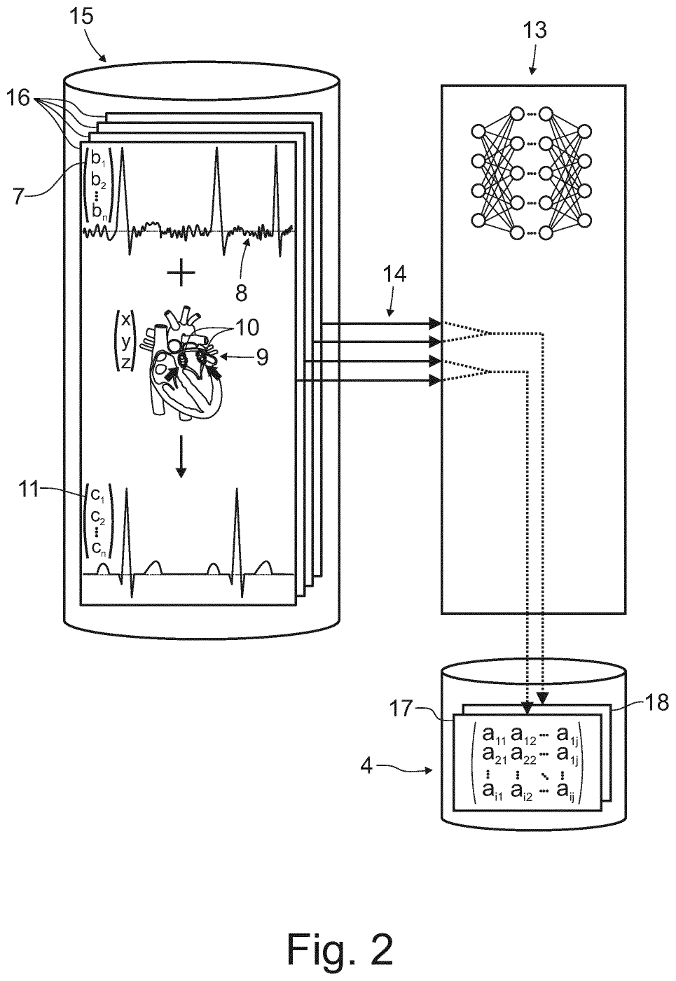

With reference to FIG. 2, the training of the machine learning model 4 will be described. Here and preferably, the trained machine learning model 4 has been or is trained in a training step 13 on a training data set 14 by the control system 2. The training data set 14 may be derived from a training database 15 that comprises ablation therapy data 16 including ablation events 9 and ECG data 8 determined prior and posterior to the ablation therapies 1 and/or one or more of the ablation events 9.

Predicting a change of electrical biomarkers 7 has the advantage that databases with a variety of ablation therapies 1 can be used for training. Therefore, preferably the training step 13 comprises retrospectively labelling the training database 15 thereby generating at least part of the training data set 14.

The labelling may in principle be performed at least partially manually. It is however preferred that the control system 2 at least partially, in particular mainly or completely, derives the electrical biomarkers 7 from the ECG data 8 of the training database 15 prior and posterior to the ablation therapies 1 and/or one or more of the ablation events 9. Automatically deriving the electrical biomarkers 7 from a database already containing ECG data 8 changes after an ablation therapy 1 enables an efficient approach for unsupervised learning.

In a preferred embodiment, the potential ablation therapy 1 includes at least two potential ablation events 9 with different ablation locations 10. In the training step 13, the control system 2 then trains a first model 17 to derive a predicted change of the electrical biomarkers 7 after applying one of the potential ablation events 9 and/or a second model 18 to derive a predicted change of the electrical biomarkers 7 of the applying potential ablation therapy. In some cases, the result of a complete ablation therapy 1 may vary from the sum of the results of single ablation events 9. In other cases, a single ablation event 9 may be the source of success of the ablation therapy 1. Looking at individual ablation events 9 and the ablation therapy 1 may therefore lead to an improved analysis.

It is noted that the training does not have to be completely automatic. The training step 13 and therefore the trained machine learning model 4 may comprise a step of automatically training and a step of manual fine-tuning. Generally, the trained machine learning model 4 may also be part of a model comprising the trained machine learning model 4 and further manually derived models.

The proposed method allows for an effective definition of a cost-function for training the trained machine learning model 4. This is advantageous as the cost function may be used with many different architectures. In one preferred embodiment, the architecture behind the machine learning itself is derived and/or changed during the training step 13. The training step 13 may include a neural architecture search and/or use guided or automated hyperparameter tuning.

For faster training of the first and the second model 17, 18, a general model may be trained and the first and the second model 17, 18 may be trained by transfer learning from the general model.

Here and preferably, in the analysis step 3 the control system 2 applies the first and the second model 17, 18 to the input data 5 to derive the predicted change of the electrical biomarkers 7. Preferably, in the analysis step 3 the control system 2 applies the first and the second model 17, 18 to a different subset of the electrical biomarkers 7 to derive a predicted sub-change of the electrical biomarkers 7 after one or more of the potential ablation events 9 and after the potential ablation therapy 1. The control system 2 can then derive the predicted change from the predicted sub-changes of the electrical biomarkers 7.

Regarding the training data set 14, the ablation therapies 1 in the training data set 14 may mainly comprise at least one focal source ablation event. Preferably, the training data set 14 may comprise ablation therapies 1 including mainly focal source ablation events. A focal source ablation event is an ablation event 9 for which a mapping or other determination of a focal source has been done previously to ablation therapy 1, to identify focal sources and where at least one of the focal sources is ablated. Nevertheless, an advantage of the proposed method is that a mapping is not always or even mostly not necessary . It is also not necessary to use the actual information on the mapping in the training data set 14, even if the training data set 14 comprises focal source ablation events derived from mapping.

The method may comprise a deriving step in which the control system 2 applies an algorithm to the ECG data 8 to derive at least part of the electrical biomarkers 7 from the ECG data 8 automatically. Preferably, in the deriving step the control system 2 applies one or more non-machine learning algorithms to the ECG data 8 to derive one or more of the electrical biomarkers 7. Additionally or alternatively, in the deriving step, the control system 2 may apply one or more secondary machine learning models to the ECG data 8 to derive one or more of the electrical biomarkers 7.

Here and preferably, the same algorithm or the same architecture is used to derive the electrical biomarkers 7 for training and in the deriving step.

The secondary machine learning models may be based on any suitable architecture, preferably on convolutional networks or an autoencoder. It is also possible to include the secondary machine learning model or models into the machine learning model 4 and apply and/or train these models together. For example, feature extraction layers are known.

The electrical biomarkers 7 may comprise at least one of: a cardiac cycle-length, an ECG morphology classification, a signal amplitude, a cardiac rhythm classification, a peak timing value, in particular R-peak timing value, a peak variability, in particular R-peak variability or a quantitative change between beats, in particular consecutive beats, for example a mean square error value. Additionally or alternatively, the input data 5 may comprise patient data, preferably at least one of age, sex, risk factors, and/or biological biomarkers, preferably biomarkers representative for blood pressure data and/or blood-measured biomarkers.

The cardiac cycle-length can be derived from an R-R-distance-measurement and/or a frequency analysis. The morphology can be identified by a score, a value like T-T-voltage increase, a comparison with QRS templates, or the like. Relevant is the standardized learning mechanism. Amplitude and power can be represented by mean values, peak values or the like. The rhythm classification may include a regularity score and/or a standard deviation of cycle-length.

Non-electrical biomarkers may be included into the machine learning mechanism to interpret the result of the prediction; they may be mixed with the electrical biomarkers 7 or included only in an output layer for example. Non-electrical biomarkers may also be used in the deriving step.

Here and preferably, the trained machine learning model 4, in particular the first model 17, is trained by using mainly the last ablation event 9 of the ablation therapies 1 of the training database 15 as the training data set 14 or by using ablation events 9 throughout the ablation therapies 1. Here, as mentioned above with respect to the definition of the term “mainly”, it would be possible to include a number of ablation events 9 that were not the last ablation events 9 in the training without creating notable adverse effects for the training data set 14.

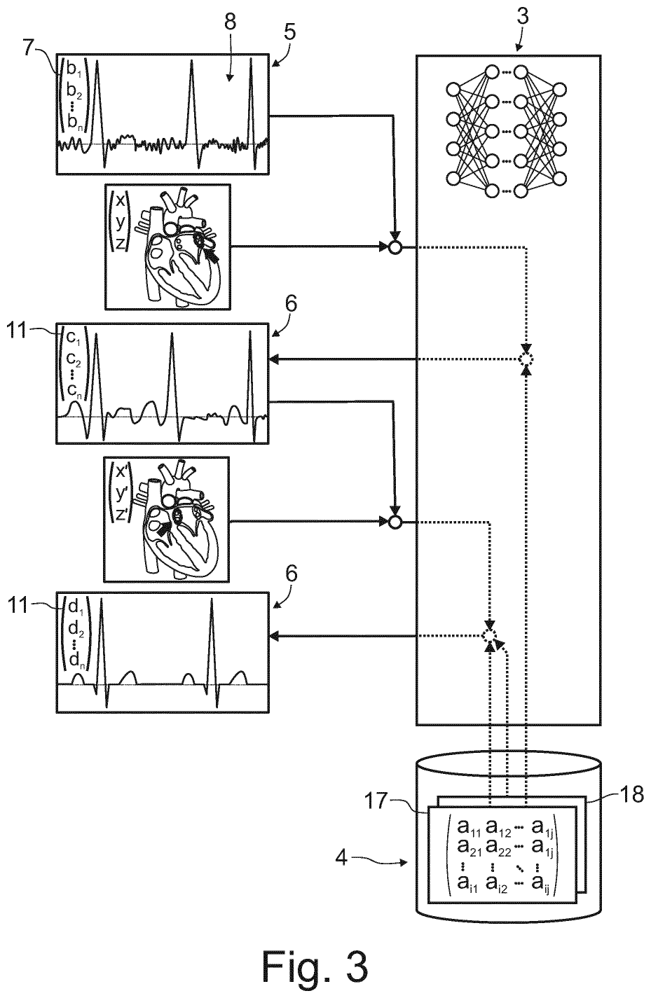

FIG. 3 shows a possible application of the first and the second model 17, 18. At first, the first model 17 is used to predict the change of the biomarkers 7 for the ablation event 9 and afterwards the first and the second model 17, 18 are used to predict the change of the biomarkers 7 after a second ablation event 9 concluding the ablation therapy 1.

The proposed method may comprise an ablation therapy classification step in which the control system 2 determines classification of one or more potential ablation events 9 and/or the potential ablation therapy 1, in particular by determining from the output data 6 a success score relating to the probability of successful treatment of the patient P by applying the potential ablation therapy 1. Preferably, the method comprises an ablation therapy determination step in which the control system 2 determines a classification for at least two potential ablation events (9) and/or at least two potential ablation therapies 1 based on the output data 6 and/or in which the control system 2 determines an optimized ablation therapy 1 based on the output data 6.

The success of a potential ablation event 9 may be classified different from the success of the potential ablation therapy 1. In particular, the control system 2 or a physician may design the potential ablation therapy by adding potential ablation events 9 targeting different electrical biomarkers. The proposed method may comprise a step of classifying potential ablation events 9 into predefined classes of potential ablation events 9. The predefined classes can then be defined as classes having one of a group of predefined results on one or more of the electrical biomarkers 7. The possibility of judging the result of a single potential ablation event 9, possibly even without the context of the complete potential ablation therapy 1, is one of the advantages of the proposed method.

The control system 2 may receive ECG data 8, preferably from an online measurement of a patient P, in particular during surgery, and output the classification of potential ablation therapies 1. Preferably the potential ablation therapies 1 are at least partly defined by measuring ECG data 8 at or near potential ablation locations 10 during surgery. In this way, it becomes possible to identify and/or verify an ablation therapy 1 during surgery. In particular, it becomes possible to include or exclude at least one potential ablation event 9 in the ablation therapy 1.

The control system 2 may receive ECG data 8 from a surgery system 19.

According to another teaching, a computer readable medium with a trained machine model 4 stored on it is proposed. Reference is made to all explanations given above. The trained machine learning model 4 on the computer readable medium has been derived in the training step 13.

According to another teaching, a control system 2 configured to perform the proposed method is proposed. Reference is made to all explanations given above.

According to another teaching, a surgery system 19 connected to or forming part of a control system 2 is proposed. The control system 2 is configured to perform the proposed method. Reference is made to all explanations given above. The surgery system 19 is adapted to be used during surgery, adapted to receive online ECG data 8 during surgery and adapted to display a result of the therapy classification step and/or the ablation therapy determination step. The surgery system may be connected to electrodes 12 for measuring the online ECG data 8. It may include hardware for processing ECG data 8, a display and/or an input unit.

1-15. (canceled)

16. A method for analyzing potential ablation therapies via a control system, wherein the method comprises an analysis step in which the control system applies a trained machine learning model to input data thereby generating output data, wherein the input data comprise electrical biomarkers derived from ECG data from a patient of at least one potential ablation therapy, wherein the potential ablation therapy includes at least one potential ablation event with a potential ablation location, wherein the output data comprises a predicted change of the electrical biomarkers after applying the at least one potential ablation therapy to the patient, and wherein the predicted change is derived from the input data via the trained machine learning model.

17. The method according to claim 16, wherein the input data is mainly constituted by ECG-derived data.

18. The method according to claim 16, wherein the input data are mainly constituted by ECG-derived data and ECG data.

19. The method according to claim 16, wherein the method further comprises analyzing the output data to identify potential ablation targets for the patient.

20. The method according to claim 16, wherein the trained machine learning model has been or is trained in a training step on a training data set by the control system, wherein the training data set is derived from a training database that comprises ablation therapy data including ablation events and ECG data determined prior and posterior to the ablation therapies and/or one or more of the ablation events.

21. The method according to claim 20, wherein the control system at least partially derives the electrical biomarkers from the ECG data of the training database prior and posterior to the ablation therapies and/or one or more of the ablation events.

22. The method according to claim 20, wherein the potential ablation therapy includes at least two potential ablation events with different ablation locations, wherein in the training step the control system trains a first model to derive a predicted change of the electrical biomarkers after applying one of the potential ablation events and/or a second model to derive a predicted change of the electrical biomarkers after applying the potential ablation therapy wherein in the analysis step the control system applies the first and/or second model to the input data to derive the predicted change of the electrical biomarkers.

23. The method according to claim 22, wherein in the analysis step the control system applies the first and the second model to a different subset of the electrical biomarkers to derive a predicted sub-change of the electrical biomarkers after one or more of the potential ablation events and after the potential ablation therapy, and wherein the control system derives the predicted change from the predicted sub-change of the electrical biomarkers.

24. The method according to claim 20, wherein the ablation therapies in the training data set mainly comprise at least one focal source ablation event.

25. The method according to claim 20, wherein the training data set comprises a plurality of ablation therapies including mainly focal source ablation events.

26. The method according to claim 16, wherein the method further comprises a deriving step in which the control system applies an algorithm to the ECG data to derive at least part of the electrical biomarkers from the ECG data automatically wherein in the deriving step the control system applies one or more non-machine learning algorithms to the ECG data to derive one or more of the electrical biomarkers, and/or wherein in the deriving step the control system applies one or more trained secondary machine learning models to the ECG data to derive one or more of the electrical biomarkers.

27. The method according to claim 16, wherein the electrical biomarkers are one or more selected from the group consisting of: a cardiac cycle-length; an ECG morphology classification; a signal amplitude; a signal power; a cardiac rhythm classification; a peak timing value; a peak variability; and a quantitative change between beats.

28. The method according to claim 16, wherein the input data further comprises patient data and/or biological biomarkers.

29. The method according to claim 20, wherein the trained machine learning model is trained by using mainly the last ablation event of the ablation therapies of the training database as the training data set or by using ablation events throughout the ablation therapies.

30. The method according to claim 16, wherein the method further comprises an ablation therapy classification step in which the control system determines a classification of one or more potential ablation events and/or the potential ablation therapy by determining from the output data a success score relating to the probability of successful treatment of the patient by applying the potential ablation therapy.

31. The method according to claim 16, wherein the method further comprises an ablation therapy determination step in which the control system determines a classification for at least two potential ablation events and/or at least two potential ablation therapies from the output data and/or in which the control system determines an optimized ablation therapy from the output data.

32. The method according to claim 31, wherein the control system receives ECG data from an online measurement of a patient during surgery, and wherein the control system outputs the classification of the at least two potential ablation therapies such that the at least two potential ablation therapies are at least partly defined by measuring ECG data at or near potential ablation locations during surgery.

33. A computer readable medium with a trained machine learning model stored on it, wherein the trained machine learning model was derived in the training step of the method according to claim 20.

34. A control system configured to perform the method according to claim 16.

35. A surgery system connected to or forming a part of a control system, wherein the control system is configured to perform the method according to claim 16, and wherein the surgery system is adapted to be used during surgery, is adapted to receive online ECG data during surgery and is adapted to display a result of the therapy classification step and/or the ablation therapy determination step.
